(19) United States
(12) Patent Application Publication
(10) Pub. No.: US 2025/0354197 A1
(43) Pub. Date: Nov. 20, 2025

(54) METHOD FOR THE ACCURATE DETERMINATION OF AGE FROM MITOCHONDRIAL DNA BY IDENTIFYING RELATIVE N6-METHYLADENINE LEVELS AT SPECIFIC SITES

(71) Applicant: VELLAB BIOTECH KFT., Szeged (HU)

(72) Inventor: Tibor VELLAI, Budapest (HU)

(73) Assignee: VELLAB BIOTECH KFT., Szeged (HU)

(21) Appl. No.: 18/873,024

(22) PCT Filed: Sep. 20, 2022
(86) PCT No.: PCT/HU2022/000017

§ 371 Date: Dec. 9, 2024

(30) Foreign Application Priority Data
Jun. 13, 2022 (HU) P2200206

(51) Int. Cl. C12Q 1/6809

(52) U.S. Cl. CPC C12Q 1/6809; C12Q 2600/112; C12Q 2600/154; C12Q 2600/158

(57) ABSTRACT

A molecular biology method, by which relative (normalized to an internal control) N6-methyladenine (6 mA) level at a selected site of the mitochondrial (mt) DNA (deoxyribonucleic acid) (mtDNA) in a tissue sample is accurately determined, and this level is projected to a reference “relative mtDNA 6 mA level-age” curve constructed earlier is described. On the reference curve, relative mtDNA 6 mA levels reversely correlate with age; the higher the relative mtDNA 6 mA level, the younger the individual analyzed. Where the value of the relative mtDNA 6 mA level cuts the reference curve, this intersection assigns the corresponding age on the X axis. The reference curve was previously established by determining 6 mA levels at a specific mtDNA site in a large (>1000) number of healthy individuals with known age. The accuracy of the measurement depends on the accuracy of the method by which mtDNA 6 mA level is determined. The method is also suitable for predicting expected lifespan in an individual with known age. The invention relies on our recent biological finding that 6 mA levels at specific mtDNA regions negatively correlate with (is reversely proportional to) the age of the individual analyzed.

The subject of the present invention is a molecular biology method, by which relative (normalized to an internal control) N6-methyladenine (6 mA) level at a selected site of the mitochondrial (mt) DNA (deoxyribonucleic acid) (mtDNA) in a tissue sample is accurately determined, and this level is projected to a reference “relative mtDNA 6 mA level-age” curve constructed earlier. On the reference curve, relative mtDNA 6 mA levels reversely correlate with age; the higher the relative mtDNA 6 mA level, the younger the individual analyzed. Where the value of relative mtDNA 6 mA level cuts the reference curve, this intersection assigns the corresponding age on the X axis. The reference curve was previously established by determining 6 mA levels at a specific mtDNA site in a large (>1000) number of healthy individuals with known age. The accuracy of the measurement depends on the accuracy of the method by which mtDNA 6 mA level is determined. Until now, there is only a single method by which relative 6 mA level at a selected genomic site can be accurately determined (patent applications entitled “A PCR-based method for the accurate determination of . . . ”, with file numbers P2100409 and W2200015). During this method, individual genomes isolated from a tissue sample examined are digested with a 6 mA-dependent restriction endonuclease, the resulting genomic fragments are then ligated to a linker DNA fragment called adapter, and finally a sequence-specific PCR- (polymerase chain reaction) based DNA amplification is achieved by using a forward primer that is simultaneously specific to the downstream part of the linker DNA fragment and the target mitochondrial genomic site adjacent to the linker. Ligation of the linker makes possible a direct amplification of the selected methylated (digested) adenine nucleobase. The quantity of the PCR product is proportional with the relative (correlated to another mtDNA site) 6 mA level determined at the given mtDNA position.

Specifically, the innovation is a molecular biology method, by which the relative 6 mA level at a specific mtDNA site in a tissue sample containing numerous individual genomes is accurately determined, and this level is then extrapolated to a reference “mtDNA 6 mA level-age” curve established earlier in order to accurately determine the age of the individual analyzed. The method is also suitable for predicting expected lifespan in an individual with known age (how long the individual will live for). The invention relies on our recent biological finding that 6 mA levels at specific mtDNA regions negatively correlate with (is reversely proportional to) the age of the individual analyzed. Thus, the epigenetic process N6-adenine methylation continuously weakens in these specific mtDNA regions during the adult lifespan, thereby serving as a solid signature of aging rate and organismal age. The method can be applied in the following major areas:
1. Age determination in forensic proceedings (age can be accurately determined from the biological trace—i.e., a tissue sample left behind—of a perpetrator with unknown identity which can significantly narrow down the number of suspected individuals, thereby promoting the success of the investigation process).
2. Expected lifespan prediction (lifespan can be predicted in an individual with known age, and in the light of this data significant changes can be established in lifestyle and in administrative—e.g., devise—issues).
3. Identifying early stages of neurodegenerative processes (the rate of the aging process differs between normal individuals and patients affected by a neurodegenerative pathology, so when the age extrapolated by the determined mtDNA 6 mA level significantly differs from the chronological age of an otherwise normal looking individual, this can indicate an early—before manifestation of cognitive decline—stage of a neurodegenerative process, and this early detection can promote the selection of a potent therapy).

### State of the Technique

### N6-Methyladenine (6 mA) Epigenetic Mark

Genetic material (genome) of living organisms consists of DNA (deoxyribonucleic acid), and in many of them the genome splits into functional units called chromosomes (a chrosomsome exists as an intact DNA chain). The DNA is built up from nucleotides (building blocks—each consists of a ribose, a phosphate group and an organics base called nucleobase), which differ from each other in the nucleobase, either adenine, guanine, cytosine or thymine. Genetic information stored in the DNA is essentially determined by the order of nucleotides—this is called DNA sequence. The human genetic information (haploid genome) consists of 3.1 billion nucleotides, which are organized into 23 pairs of chromosomes, 22 pairs of autosomes and 1 pair of sex chromosomes.

The functional units of chromosomes are called genes, the products (proteins and RNAs—ribonucleic acids) of which operate the cells. At the DNA level, the activity of genes can be changed by two major ways. First, at the genetic level—in this case certain nucleotides become modified (lost, added or transformed into another nucleotide). Such changes involve mutations and genetic polymorphisms. Second, at the epigenetic level—in this case the nucleotide order remains intact but a small chemical alteration, addition of a methyl group (—CH3), affects a specific nucleobase, adenine or cytosine. Adenine can primarily be converted into N6-methyladenine (6 mA), while cytosine can primarily be converted into 5-methylcytosine (5mC) (FIG. 1). In general, 6 mA has a gene-activating role (where the modification takes place in the genome, the affected gene becomes activated), whereas 5mC has a role in gene repression (X chromosome inactivation, genomic imprinting and maintaining genomic stability by repressing transposable elements called “jumping genes”). These epigenetic modifications are often triggered by environmental factors and can be inherited into the daughter cells and subsequent generations. 6 mA mark is generated from adenine, and the process is catalyzed by a DNA methyladenine transferase enzyme (FIG. 1). 6 mA can be re-converted into adenine by the action of a 6 mA demethylase enzyme (FIG. 1).

DNA N6-adenine methylation has been widely observed in plants and bacteria. The process was recognized in animal and organellar (mitochondrial) genome just a few years ago (Greer et al., 2015; Zhang et al., 2015; Wu et al., 2016; Hao et al., 2022). Recent studies however suggest that the presence of 6 mA in animal and organellar genomes is only a result of methodological artifacts (Schiffers et al., 2017; O'Brown et al., 2019; Douvlataniotis et al., 2020). Nevertheless, the biological functions of 6 mA mark remain largely unexplored.

### Techniques Demonstrating DNA Methylation (Epigenetic Mark)

Several techniques have been developed to demonstrate the presence of methylated DNA nucleobases (6 mA and 5mC epigenetic marks). They include single molecule real-time sequencing (SMRT-seq), bisulfite sequencing (the genome is treated by Na-bisulfite that converts cytosine nucleobases into uracil nucleobases, but 5mC remains unmodified by this agent), liquid chromatography-tandem mass spectrometry (LC-MS/MS) and labeling (hybridization) with a 6 mA/5mC-specific antibody (Dahl and Guldberg, 2003; Flusberg et al., 2010; Rocha et al., 2010). These methods are rather costly (as they rely on expensive instruments and high expertise), time consuming and hardly available (e.g., SMRT-seq is provided by only a few companies worldwide), thus their diagnostic application has not yet essentially been established in medicine and forensic genetics. Their largest disadvantage, however, is that they frequently generate artifact (O'Brown et al., 2019; Schiffers et al., 2017; Douvlataniotis et al., 2020). This mainly comes from the fact that 6 mA and 5mC epigenetic marks widely present in the genome of bacteria that often infect eukaryotic tissues and also in eukaryotic RNA. Artifact can also be generated when the specificity of antibodies against 6 mA and 5mC is insufficient, and by technological limitations of SMRT-seq. Furthermore, these techniques listed above are not capable of identifying the quantity (relative level) of a methylated nucleobase at a given genomic site in a tissue sample analyzed (genomic DNA is isolated from a large number of cells), i.e., how many individual genomes are methylated at a given genomic site in the sample, rather they can only provide “yes” or “no” answer to the methylation status of the nucleobase examined.

Alternatively, 5mC levels can be determined by using 5mC-dependent/sensitive restriction endonucleases and a subsequent PCR-based amplification of the target site (Luo et al., 2016; Yao et al., 2017). This method, however, is also unable to accurately determine methylation level at a certain genomic site in a tissue sample. Thus, these DNA methylation detection technologies listed above still remain largely unused in medical and forensic applications. A solution was provided recently by a novel method, during which genomic DNA isolated from a tissue sample is digested with a 6 mA- or 5mC-specific restriction endonuclease, the resulting DNA fragments are then ligated to a linker DNA fragment called adapter, and the target site is amplified by a PCR reaction using a forward primer that is simultaneously specific to both linker sequence (10-15 nucleotides) and adjacent genomic sequence (10-15 nucleotides). The application of the linker DNA fragment makes it possible that the digested (methylated) DNA sequence is directly amplified. This is crucial because methylation of an adenine nucleobase at the N6 position is a rather rare event, and only a very few individual (cellular) genomes in a tissue sample are methylated at a given time. Using this method, the PCR product trustworthily reflects the relative 6 mA or 5mC level of the selected genomic site (patent applications: “A PCR-based method for the accurate determination of . . . ”; file numbers: P2100409 and W2200015—the inventor gave us a permit to look at the application content under the condition of maintaining strictly the IP rights-NDA). The more genomes are methylated at a selected genomic site (nucleobase) among the individual genomes (tissue sample) analyzed, the higher quantity of the PCR product generated.

### DNA Methylation in Age Determination

In the last decade, it has been shown that the epigenetic mark 5mC is formed in an age-depending manner in the human genome. 5mC primarily accumulates in transposable element (TE) loci during early development, and plays an important role in the repression of these mutagenic sequences, thereby maintaining genomic stability (Yoder és mtsi., 1997). Dr. Steve Horvath (UCLA, US) has been developed an algorithm, by which the biological age of a human tissue sample can be determined through identifying 5mC contents of certain genomic regions—“Horvath's clock” (Horvath, 2013). He demonstrated that 5mC content in these genomic regions decreases with age. Determination of 5mC content in specific genomic regions is basically performed by bisulfite sequencing, and the result provided is used to determine biological age with error means of 7-10 years (±7-10 years), which is not precise enough to apply the technology in medicine and forensic genetics requiring a large accuracy. Although this method is used by several companies (e.g., Chronomics, Altos Labs, Zymo Research, Elysium Health and Ra Pharmaceuticals Ltd.) in the field of DNA diagnostics, results are only informative for layman procurers. Moreover, it has been turned out that 5mC levels grow at certain genomic positions, but decrease at other genomic positions, during lifespan, and that the 5-cytosine methylation process is highly affected by environmental and physiological factors. Lastly, there are organisms, such as the nematode Caenorhabditis elegans and fruit fly Drosophila melanogaster, that essentially lack the phenomenon of 5-cytosine methylation. In the light of these facts, one can state that identifying 5mC levels at certain genomic regions is not accurate enough to accurately determine (biological) age of an individual.

### The Problem that is Solved by the Innovation

We have shown from human mitochondrial DNA (mtDNA) samples that relative 6 mA levels at specific sites decrease with age during the adult lifespan (FIG. 2). Although at present we do not know the biological meaning of this phenomenon, 6 mA epigenetic mark at specific mtDNA sites is potent marker of biological age. This invention relies on the observation that relative 6 mA levels at specific sites of the mitochondrial genome gradually lower as the organism ages. According to our knowledge, such a finding is not available in the literature, Using specific primer pairs, we determined relative 6 mA levels at multiple sites of the mitochondrial genome, and found a gradual, age-dependent decrease at each site analyzed.

Later, we determined relative 6 mA level at a specific site of the mtDNA in a large number (several hundreds) of healthy individuals with known identity (age), and constructed a reference “relative mtDNA 6 mA level-age” curve, in which 6 mA levels show an invers correlation with age (FIG. 3). The lower the 6 mA level determined, the older the individual analyzed. This data set was termed as a reference “relative mtDNA 6 mA level-age” curve. If relative 6 mA level at this specific site is determined in the mtDNA of an individual with unknown identity, and the value (on the Y axis) obtained is projected to the reference curve, then the intersection projected to the X axis at right angles assigns the age on the individual (FIG. 3). The method yields relatively exact (error means of 2-3 years) and specific (no artifact) data, fast (PCR-based) and cost effective, so it can be used to establish epigenetic DNA diagnostics, which is a potential important branch of the DNA diagnostics industry.

The method can be applied in the following major diagnostic fields:
1. Forensic genetics. In the field of criminal acts, biological trace (evidence—e.g., a hair or blood drop) from the perpetrator can often be collected. In such cases, genomic DNA is isolated from the trace left behind to determine the DNA profile of the perpetrator. The profile is frequently based on individual DNA polymorphisms (e.g., repetition number of microsatellites called short DNA repeats). Then, the result (profile) is compared with a criminal DNA database containing DNA profiles of known perpetrators, and if there is a match the identity of perpetrator can be identified. In most cases, however, the DNA database does not contain a profile that would match with the actually identified DNA polymorphism (i.e., the profile of the perpetrator has not yet been introduced into the DNA database). In such cases, identifying relative 6 mA level at a specific mtDNA locus from the biological trace and comparing the value with the reference “relative mtDNA 6 mA level-age” curve, the age of the perpetrator can be determined accurately (for example, 56±2 years—so, with a 2-year of error limit) (FIG. 4). In the light of the result determined, the number of suspects can be narrow down significantly which can largely promote the success of the investigation process.
2. Lifespan prediction in individuals with known age. Different individuals of a species live for various times. For example, if one randomly chooses three 50 years old persons, theoretically the first will live for 56 years, the second will live for 66 years while the third will die at age 84. So, lifespan in individuals with the same age can vary significantly. Using the method shown above, the expected lifespan of an individual with known age can be predicted (how long she or he will live for). If the relative 6 mA level at a given mtDNA site in a tested person is significantly higher than those expected from the reference curve at a given age, the value predicts a shorter lifespan then it is expected. So, the higher the relative mtDNA 6 mA level at a given age, the shorter the expected lifespan of the individual. It is important to note that this is “only” a prediction because the person can be die much earlier than it is expected from her/his 6 mA level, due to, for example, a fatal accident. Those getting an unfavorable prediction (a short lifespan expectancy) by the test can change their lifestyle (e.g., starting physical activity, stress-avoidance and better nutrition) significantly in order to reverse the rate at which the cells age. In addition, an unfavourable expectancy can also initiate testament preparation.
3. Early detection of neurodegenerative processes. If the relative mtDNA 6 mA level identified in a person with known age significantly differs from those expected from the reference curve at the given age, then the value may indicate an early stage (prior to the manifestation of cognitive decline) of a neurodegenerative process (FIG. 5). This is because the rate of the aging process in a person affected by a neurodegenerative disease (e.g., Alzheimer's, Parkinson's or Huntington's disease, or ALS) differs (generally faster) from those found in non-affected persons at the same age, and the difference can be accurately measured by the method introduced in this patent application (the higher the relative mtDNA 6 mA level at a given age, the higher the rate at which the aging process takes place). Relative 6 mA levels at specific mtDNA sites thus can be used as an early marker of various neurodegenerative pathologies. This is a significant finding because when a neurodegenerative disease becomes manifested (obvious cognitive problems), then major parts of the brain is already affected by neuronal demise, so pharmacological treatments applied prove to be largely ineffective (neurons that have already died cannot be revitalized). A solution can be provided by an early (well before the manifestation of cognitive decline) diagnosis, but a suitable marker for doing this has not yet been generated. When relative mtDNA 6 mA level indicates involvement in an otherwise normal looking (healthy) individual, then it is suggested for the patient to be subjected to a further PET (positron emission tomography) or NMR (nuclear magnetic resonance) examination, and, by recognizing the presence of a degenerative process, to a medical therapy.

### DETAILED DESCRIPTION OF THE INNOVATION

To determine age from mtDNA, the following steps have to be performed: i) identification of relative 6 mA level at a given mtDNA site in a tissue sample obtained from a person with unknown identity by a PCR-based approach (semi-quantitative PCR or real-time quantitative PCR); ii) extrapolation of the 6 mA level identified (the Y axis) assigns a value on a reference “relative mtDNA 6 mA level-age” curve; iii) projection of this value (intersection) to the X axis assigns the age of the individual. To perform accurately this set of experiments, only a single method is available so far, with the following steps: i) isolation of genomic DNA from a tissue; ii) digestion of genomic DNA with a 6 mA-specific restriction endonuclease (e.g., DpnI that cuts the DNA at the -GATC- sequence only when A is methylated: -GA^{Me}TC-); iii) ligation of a linker DNA fragment to the digested genomic DNA fragments; iv) PCR amplification of the target (digested) site by using a forward primer that is simultaneously specific to both linker and adjacent mtDNA sequence; v) quantification of the PCR product; vi) comparing the quantity of the product with that of an internal control (this normalization leads to a relative 6 mA level). In this case, the PCR product trustworthily reflects relative 6 mA level at the selected mtDNA site. This method is described in recent patent applications entitled “A PCR-based method for the accurate determination of . . . ”; with file numbers P2100409 and W2200015. The owner of this application (inventor) gave us a permit to look at the file (protocol) under condition of maintaining every aspect of his IP rights.

Using this technique, we previously identified relative 6 mA levels at a specific mtDNA site (at a given adenine nucleobase) in many hundreds of individuals with known age (identity). From data obtained, we established a reference “relative mtDNA 6 mA level-age” curve. The persons analyzed were healthy and had various ages. Then, relative mtDNA 6 mA level identified in a person with unknown identity was projected to the reference curve, and the intersection assigned the age of the individual on the axis X (FIG. 4). Based on our measurements, deviation of data was within the margin of error of 2-3 years (±2-3 years). If relative 6 mA level at a human mtDNA site is identified in an individual with known identity (age), and the value obtained significantly differs from that the reference curve otherwise indicates at a given age, then the difference may reflect the presence of an early stage of a neurodegenerative process (FIG. 5). In this case, the affected individual is suggested to undergo an imaging examination such PET or NMR.

### A Construction Example

Method (its technical basis is described in the patent applications entitled “A PCR-based method for the accurate determination of . . . ”; file numbers: P2100409 and W2200015, and the inventor gave us his permit to look at the file (protocol) under condition of maintaining every aspect of IP rights-Non-Disclosure Agreement):

Isolation of Genomic DNA from Samples

Isolation from blood samples was performed by using Thermo Scientific GeneJET Genomic DNA Purification Kit (#K0721).

Digestion of Genomic DNA with DpnI Restriction Endonuclease

Add the following components into an Eppendorf tube:
1 μl DpnI enzyme (10 U/μl, ThermoFisher Scientific, ER1701)
4 μl Tango Buffer (10×)
15 μl H₂O
20 μl genomic DNA solution (minimum at 10 ng/μl concentration)
(final volume: 40 μl/sample)
→incubate at 37° C. for 20 min
Inactivation of DpnI: incubate at 80° C. for 20 min

### Ligation of Linker DNA to the Digested Genomic DNA Fragments

Add the following components to the inactivated mixture (40 μl):
4 μl T4 ligase (5 U/μl, ThermoFisher Scientific, EL0011)
5 μl ATP (2 mM) (ThermoFisher Scientific, 100 mM, R0441)
5 μl genomic linker (at 100× concentration)
4 μl Tango Buffer
22 μl H₂O
Final volume of the mixture: 80 μl
Incubate samples at 4° C. for overnight
Inactivation of ligase: incubate samples at 80° C. for 20 min

DNA sequence of the linker:
5′-TAG ATC TGA CCT AAC GGT AAG AGA GTT
TCA TAA TAt ttt ttt ttt ttt ttT AT-TAT GAA ACT
CTC TTA CCG TTA GGT CAG ATC TA-3'

### PCR Reaction

6mA samples (DpnI)-
Forward (left) primer:
5′-ATG CAC TAC TCA CCA GAC GC-3′,
reverse (right) primer:
5′-TCC CAA TGT ATG GGA TGG CG-3′

### Reaction Mixture:

10 μl ABI master mix (2×)
3.5 μl digested/ligated DNA solution (template)
3 μl primer mix (5-5 μM)
3.5 μl H₂O
Final volume: 20 μl/sample

### PCR Condition (DpnI):

1. initial denaturation: 95° C., 30 sec
2. denaturation: 95°, 10 sec
3. annealing and extension: 60° C., 45 sec
4. repeat steps 2, and 3. with 37×
5. store samples at 4° C.

Control (PvuII)—forward (left) primer: 5′-acc gcc atc ttc agc aaa c-3′ and reverse (right) primer: 5′-ttt cat ctt tcc ctt geg gta c-3′

### Reaction Mixture:

10 μl ABI master mix (2×)
1 μl DNA solution (template)
3 μl primer mix (5-5 μM)
6 μl H₂O
Final volume: 20 μl

### PCR Condition (PvuII):

1. initial denaturation: 95° C., 60 sec
2. denaturation: 95°, 10 sec
3. annealing and extension: 60° C., 45 msp
4. repeat steps 2, and 3. by 27 times
5. store samples at 4° C.

### Gel Documentation

run samples on 1% agarose gel, 80 mV run, photo by Kodak camera

### Novelty of the Invention

1. Recognition that relative 6 mA levels at specific sites of the mitochondrial genome grow proportionally with age. Based on this finding, age can be accurately determined from mtDNA isolated from an individual with unknown identity.
2. Recognition that relative 6 mA levels at specific mtDNA sites are predictive for lifespan.
3. Recognition that relative 6 mA levels at specific mtDNA sites are indicative for early stages of neurodegenerative processes.

### REFERENCES

Dahl C, Guldberg P. DNA methylation analysis techniques. Biogerontology 4, 233-250 (2003). PMID: 14501188
Douvlataniotis K, Bensberg M, Lentini A, Gylemo B, Nestor C E. No evidence for DNA N 6-methyladenine in mammals. Sci. Adv. 2020 Mar. 18; 6 (12): eaay3335. PMID: 32206710
Flusberg B A, Webste D R, Lee J H, Travers K J, Olivares E C, Clark T A, Korlach J, Turner S W. Direct detection of DNA methylation during single-molecule, real-time sequencing. Nat. Methods 7, 461-465 (2010). PMID: 20453866
Greer E L, Blanco M A, Gu L, Sendinc E, Liu J, Aristizábal-Corrales D, Hsu C-H, Aravind L, He C, Shi Y. DNA Methylation on N6-Adenine in C. elegans. Cell 161, 868-878 (2015). PMID: 25936839
Hao Z, Wu T, Cui X, Zhu P, Tan C, Dou X, Hsu K W, Lin Y T, Peng P H, Zhang L S. et al. (2020) N(6)-deoxyadenosine methylation in mammalian mitochondrial DNA. Mol. Cell 78, 382-395 e388. PMID: 32183942
Horvath S. DNA methylation age of human tissues and cell types. Genome Biol. 2013; 14 (10): R115. PMID: 24138928
Luo G-Z, Wang F, Weng X, Chen K, Hao Z, Yu M, Deng X, Liu J, He C. Characterization of eukaryotic DNA N(6)-methyladenine by a highly sensitive restriction enzyme-assisted sequencing. Nat. Commun. 7, 11301 (2016). PMID: 27079427
O'Brown Z K, Boulias K, Wang J, Wang S Y, O'Brown N M, Hao Z, Shibuya H, Fady P-E, Shi Y, He C, Megason S G, Liu T, Greer E L. Sources of artifact in measurements of 6 mA and 4mC abundance in eukaryotic genomic DNA. BMC Genomics 2019 Jun. 3; 20 (1): 445. PMID: 31159718
Rocha M S, Castro R, Rivera I, Kok R, Smulders Y M, Jakobs C, de Almeida I T, Blom H J. Global DNA methylation: comparison of enzymatic- and non-enzymatic-based methods. Clin. Chem. Lab. Med. 48, 1793-1798 (2010). PMID: 20979561
Schiffers S, Ebert C, Rahimoff R, Kosmatchev O, Steinbacher J, Bohne A-V, Spada F, Michalakis S, Nickelsen J, Müller M, Carell T. Quantitative LC-MS Provides No Evidence for m 6 dA or m 4 dC in the Genome of Mouse Embryonic Stem Cells and Tissues. Angew Chem Int Ed Engl. 2017 Sep. 4; 56 (37): 11268-11271. PMID: 28371147
Wu T P, Wang T, Seetin M G, Lai Y, Zhu S, Lin K, Liu Y, Byrum S D, Mackintosh S G, Zhong M, Tackett A, Wang G, Hon L S, Fang G, Swenberg J A, Xiao A Z. DNA methylation on N(6)-adenine in mammalian embryonic stem cells. Nature. 2016 Apr. 21; 532 (7599): 329-33. PMID: 27027282
Zhang G, Huang H, Liu D, Cheng Y, Liu X, Zhang W, Yin R, Zhang D, Zhang P, Liu J, Li C, Liu B, Luo Y, Zhu Y, Zhang N, He S, He C, Wang H, Chen D. N6-methyladenine DNA modification in Drosophila. Cell 161, 893-906 (2015). PMID: 25936838
Yao B, Cheng Y, Wang Z, L Y, Chen L, Huang L, Zhang W, Chen D, Wu H, Tang B, Jin P. DNA N6-methyladenine is dynamically regulated in the mouse brain following environmental stress. Nat. Commun. 8, 1122 (2017). PMID: 29066820
Yoder J A, Walsh C P, Bestor T H. Cytosine methylation and the ecology of intragenomic parasites. Trends Genet. 1997 August; 13 (8): 335-40. PMID: 9260521

### FIGURE LEGENDS

FIG. 1. N6-methyladenine (6 mA) epigenetic mark. Adenine (A) can be converted to N6-methyladenine (6 mA) by the addition of a methyl group (—CH3). The —CH3 group is transferred to the N atom at position 6 of adenine by a DNA N6-adenine methyltransferase enzyme. A N6-methyladenine demetallize enzyme can remove the —CH3 group from 6 mA, thereby re-generating an adenine. Thus, the methylation and demethylation processes together influence the methylation status of a single adenine at a given genomic site. The —CH3 group is highlighted.

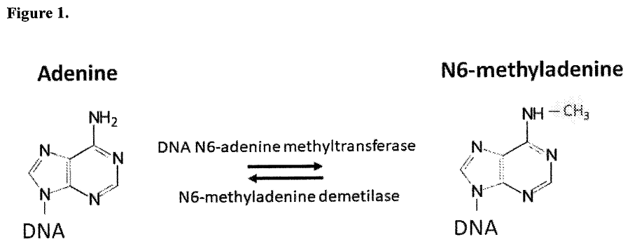

FIG. 2. Relative N6-methyladenine (6 mA) level at a specific mitochondrial DNA (mtDNA) site gradually decreases with age. a) Gel photo showing relative 6 mA levels at a specific mtDNA site in patients with age of 22, 35, 58 and 68 years (PCR products). Top: control bands, bottom: 6 mA levels. Quantities of control bands are nearly similar among the samples (similar quantities of template mtDNA were used for the analysis). a′) Quantification of bands show in panel (a). Samples were normalized to their corresponding controls. b) Relative mtDNA 6 mA levels of individual with different ages. 6 mA levels proportionally decrease with age. Top: control bands, bottom: 6 mA levels of samples.

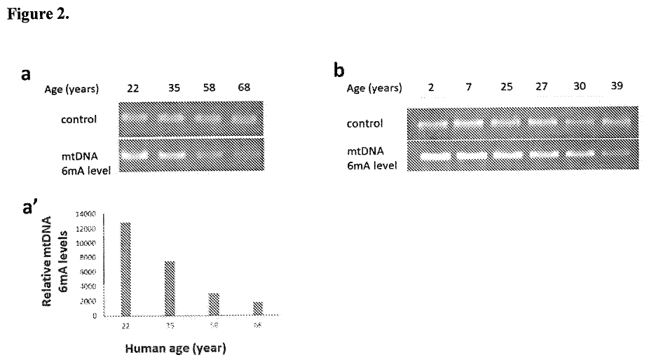

FIG. 3. Prediction of human lifespan by identifying relative 6 mA level at a specific mtDNA site. The reference “relative 6 mA level-age” curve (light blue line) was constructed by determining 6 mA level at a specific mtDNA site in individuals with known identity (age). When relative 6 mA level (dotted red line) at this mtDNA site is determined in a person with unknown identity, and the value obtained is projected to the reference curve, the intersection (projected to the X axis) assigns the age of the individual (e.g., 80 years).

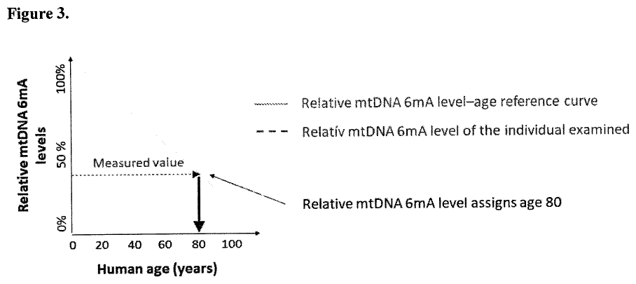

FIG. 4. Lifespan prediction in an individual with known age by determining relative N6-methyladenine (6 mA) level at a specific mitochondrial DNA (mtDNA) site. Determination of relative 6 mA level at a specific mtDNA site can be used to predict the lifespan of the individual analyzed. Two gel images showing 6 mA levels in persons with different ages. Samples with ages 60, 62, 64, 65, 66, 68, 77, 77 and 85 years display levels that correspond to those found in the reference curve at the same ages. Samples with ages 72, 76 and 80 show 6 mA levels that are much higher than those expected from the reference curve at the same ages (indicated by a yellow “!” mark). These individuals are predicted to live much longer than the average at the same ages. Samples with ages 74 and 82 exhibit much lower 6 mA levels than those found on the reference curve at the same ages. These samples are indicated by a red “!!” mark). These persons are predicted to live much shorter than the corresponding average at the same ages.

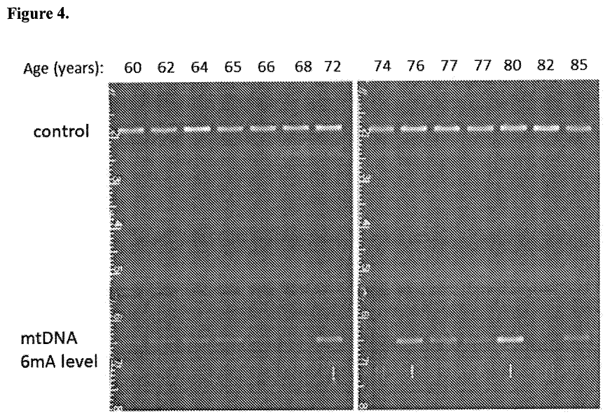

FIG. 5. Predicting early stages of neurodegenerative processes by the determination of relative N6-methyladenine (6 mA) level at a specific mitochondrial DNA (mtDNA) site. The aging rate in patients affected by a neurodegenerative disease differs from those found in age-matched healthy controls. If relative 6 mA level (dotted red line) at a specific mtDNA site is determined in a person with known age (50), and this level significantly differs from that indicated by the reference curve (blue line) at the same age (50), thereby projecting, for example, an age of 70 years, then the 6 mA value indicates the presence of a neurodegenerative process.

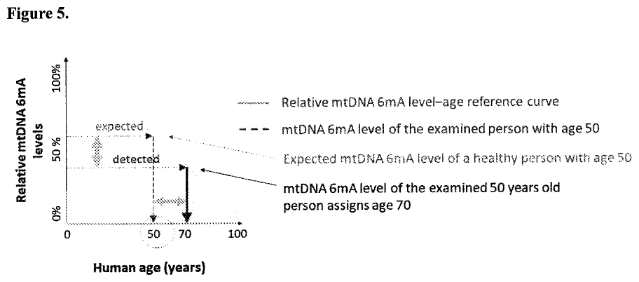

1. Method/procedure for an accurate determination of age from mitochondrial DNA (mtDNA) by identifying relative N6-methyladenine (6 mA) levels at specific sites of the mtDNA.

2. Method/procedure according to claim 1, when the organism analyzed is any eukaryotic species.

3. Method/procedure according to claim 1, when the determination of relative 6 mA levels at specific mtDNA sites is achieved by any suitable molecular technique.

4. Method/procedure according to claim 1, when the target site is an optional mtDNA sequence.

5. Method/procedure according to claim 1, when the type of the examined tissue sample is optional.

6. Method/procedure according to claim 1, when the population in which the determination of relative 6 mA levels at specific mtDNA sites is achieved is optional.

7. Method/procedure according to claim 1, when the aim of the analysis is to determine age of an individual with unknown identity (age).

8. Method/procedure according to claim 1, when the aim of the analysis is to predict lifespan in an individual with known age.

9. Method/procedure according to claim 1, when the aim of the analysis is to identify early stages of a neurodegenerative process.
